(19) United States
(12) Patent Application Publication
(10) Pub. No.: US 2006/0078460 A1
(43) Pub. Date: Apr. 13, 2006

(54) Anion generator for incorporation into lighting apparatuses and other appliances

(76) Inventors: Jason Ryu, Palisades Park, NJ (US); Chang-Min Lee, Seodaemun-Ku (KR)

Correspondence Address: RYNDAK & SURI LLP, 200 W MADISON STREET, SUITE 2100, CHICAGO, IL, 60602, US

(21) Appl. No.: 11/097,767

(22) Filed: Apr. 2, 2005

Related U.S. Application Data
(60) Provisional application No. 60/617,826, filed on Oct. 12, 2004.

(51) Int. Cl. A61L 9/22; A61L 9/18

(52) U.S. Cl. 422/5; 422/121; 422/22

(57) ABSTRACT

A negative ion generator for improving the air quality of a room or space and may be conveniently incorporated into a lighting apparatus, appliance or other house item. The anion generator includes a metal fiber brush which reduces or eliminates ozone production. The lighting apparatus may take a variety of forms and may also include a photocatalyst coating to further purify the air.

### RELATED APPLICATION

This application claims the benefit of U.S. Provisional Application No. 60/617,826, filed Oct. 12, 2004.

### FIELD OF THE INVENTION

The present invention relates to an ionizer apparatus for generating anions and purifying air, and more particularly to an ionizer apparatus incorporated into a lighting device or appliance for preventing generation of ozone while generating anions and purifying air, in addition to lighting a room or providing another function of the appliance.

### SUMMARY OF THE INVENTION

Negative ions or anions have the effects of purifying blood, activating cells, increasing immunity, controlling the autonomic nervous system, removing toxic materials generated by electromagnetic waves, eliminating dust and bacteria, adjusting humidity and removing odor. When a person inhales such anions, there occur the beneficial effects of promoting the metabolism of cells, purifying blood, tranquilizing nerves, relieving fatigue and stimulating appetite. Under fresh natural conditions, it is known that the number of anions per 1 cc of air is 800-2,000, for example, in forests, near hot springs or waterfalls, and in seashore areas. Indoor air, on the other hand, often contains various contaminants such as dust, smoke, volatile organic compounds (VOCs) emitted by building materials and furnishings, bacteria, viruses, mold and the like. These contaminants tend to be positively charged as cations which deplete the density of anions in the air and lead to uncomfortable breathing and health risks. Anion generators can restore the balance of positive and negative ions, but conventional anion generators suffer from a number of problems. For example, they produce ozone and emit electromagnetic radiation, both of which can be harmful and are undesirable, and they require relatively high electric energy to operate.

In a first embodiment there is provided a lighting apparatus for purifying the air without a UV lamp. The lighting apparatus uses a metal fiber brush which generates anions but reduces or eliminates the generation of ozone. The lighting apparatus also prevents anions generated from the anion generator from being removed by electromagnetic interference. The brush includes numerous metal fibers, each of the metal fibers being formed on an outer surface thereof with numerous tiny projections.

In another embodiment, an ionizer apparatus is incorporated at the ends of fluorescent tubes.

In another embodiment, a night light is provided which incorporates an ionizer apparatus in a multi-functional item that can produce fresh air at inexpensive costs as well as provide light. The night light is provided with one or more light emitting diodes (“LEDs”) instead of incandescent or fluorescent bulbs. The use of LEDs reduces power consumption, extends the lifespan of the light sources, reduces the maintenance costs of the lighting apparatus, and is also a safer lighting source since less heat is generated. Additionally, the use of LEDs provides for control of the amount of light generated as well as the ability to provide different color LEDs and thus provide a variety of aesthetics to a room.

In another embodiment, a light bulb of the lighting apparatus may be coated with titanium dioxide, which acts as a photocatalyst when activated by light from the light bulb, to eliminate contaminants in the ambient air.

In another embodiment, an ionizer apparatus is incorporated into a photograph frame. The photograph frame may also incorporate one or more LEDs.

In another embodiment, there is provided a ballast for a fluorescent lamp which incorporates a negative ion generator with a metal brush.

In still another embodiment, an ionizer apparatus is incorporated into a light stand, such as a desk light stand or floor lamp.

In yet another embodiment of the invention, the ionizer apparatus is incorporated into a fan, such as a portable fan or ceiling fan.

In yet another embodiment of the invention, the ionizer apparatus is incorporated into a portable audio device such as an MP3 player.

In yet another embodiment of the invention, the ionizer apparatus is incorporated into a television set.

In yet another embodiment of the invention, the ionizer apparatus is incorporated into a computer such as a desktop computer or a portable laptop computer.

In yet another embodiment of the invention, the ionizer apparatus is incorporated into an air conditioner or heater.

In yet another embodiment of the invention, the ionizer apparatus is incorporated into a telephone or cellular telephone.

In yet another embodiment of the invention, the ionizer apparatus is incorporated into a hair dryer.

In yet another embodiment of the invention, the ionizer apparatus is incorporated into a clock.

In yet another embodiment of the invention, the ionizer apparatus is incorporated into a necklace or pendant.

In yet another embodiment of the invention, the ionizer apparatus is incorporated into the passenger compartment of an automobile, for example, in the dashboard of the automobile.

### BRIEF DESCRIPTION OF THE FIGURES

FIG. 1a is an exploded prospective view showing a lighting device for generating anions and purifying air according to the present invention;

FIG. 1b is a perspective view showing an embodiment of a lighting device for generating anions and purifying air according to the present invention;

FIG. 1c is a sectional view showing a lighting device for generating anions and purifying air according to the present invention;

FIG. 1d is a sectional view showing another embodiment of a lighting device according to the present invention;

FIG. 2 illustrates a negative ion generator and includes a partial sectional view of a metal fiber brush which emits anions for purifying air;

FIG. 3 is a circuit diagram of a lighting device for generating anions and purifying air according to the present invention;

FIG. 4 is a perspective view of a fluorescent tube having negative ion generators according to one embodiment of the present invention;

FIG. 5 is an exploded perspective view of the negative ion generating nightlight;

FIG. 6 is a front perspective view of a negative ion generating nightlight illustrated in FIG. 5;

FIG. 7 is a rear perspective view of the negative ion generating nightlight illustrated in FIG. 5;

FIG. 8 is a front perspective view of a picture frame having a negative ion incorporated therein;

FIG. 9 is a side view of the ballast assembly for generating negative ions and retaining a circular fluorescent light bulb;

FIG. 10 is a bottom view of the ballast illustrated in FIG. 9;

FIG. 11 illustrates a desk lamp having a negative ion generator;

FIG. 12 illustrates a fan having a negative ion generator;

FIG. 13 illustrates a portable audio device having a negative ion generator;

FIG. 14 illustrates a television set having a negative ion generator;

FIG. 15 illustrates a personal computer having a negative ion generator in the CPU case and the monitor;

FIG. 16 illustrates a telephone having a negative ion generator;

FIG. 17 illustrates a hair dryer having a negative ion generator;

FIG. 18 illustrates a necklace having a pendant with a clock and a negative ion generator;

### DETAILED DESCRIPTION

A conventional anion generator includes an anion generating plate, a plurality of electron guns, an electronic plate having a plurality of holes and an anion collecting panel. The anion generator receives a high voltage from an amplifier and emits electrons to the electron guns mounted within the anion generator. The electrons discharged from the electron guns strike against the electron plate and the generate anions. The generated anions are discharged to the outside of the housing through the holes of the electronic plate. A problem with this type of anion generator is that it also produces ozone, which can be irritating in small quantities and lethal in larger concentrations. Other problems with conventional ion generators are that they generate electromagnetic radiation and require relatively high electric energy to operate. They also are often fairly large and inconvenient and they do not produce cluster ions which are the most desirable type.

Accordingly, it is desirable to provide an anion generator which produces beneficial anions but reduces or eliminates the generation of ozone, and may be conveniently incorporated into a lighting device or appliance.

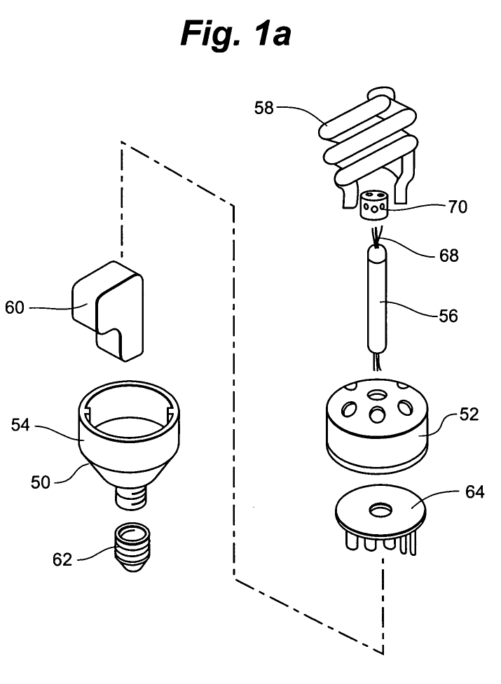

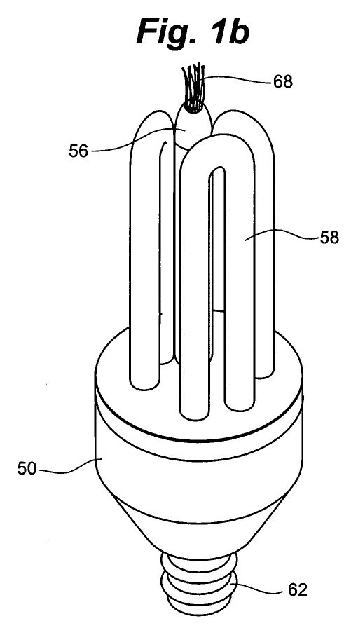

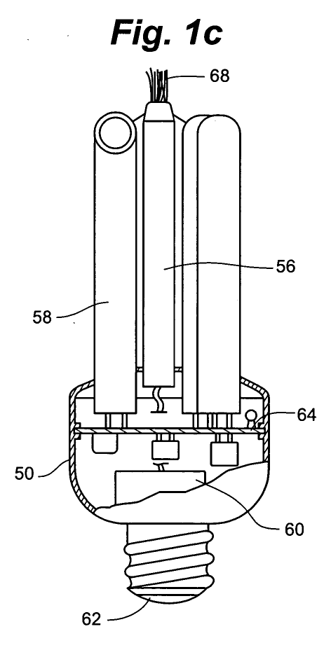

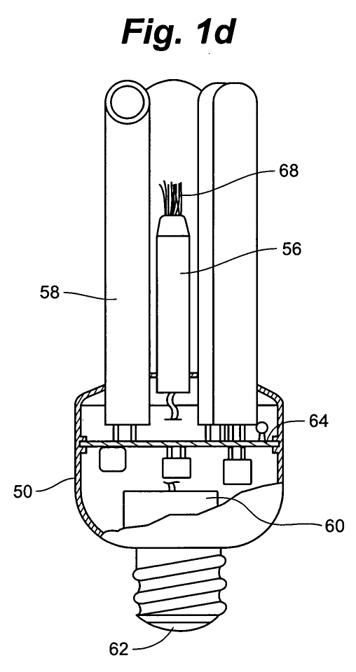

Referring to FIGS. 1a to 1d, a lighting apparatus according to the present invention comprises a cylindrical housing 50 consisting of an upper part 52 and a lower part 54, an anion generator 56, a lamp 58 mounted on upper part 52 of housing 50; and an anion electric power source 60 engaged with a socket 62 within lower part 54 of housing 50. The anion electric power source 60 amplifies power to high voltages and applies the high voltages to the anion generator 56. Further, the electric power source 60 is fixedly mounted within upper part 52 of housing 50. The anion electric power source 60 is sealed within a case to prevent electromagnetic interference generated from the anion electric power source 60 from affecting a lamp electric power source 64 applying power to lamp 58. The invention is not limited to the spiral lamp configuration as illustrated in FIG. 3a, and other types of lamps and/or a plurality of lamps are contemplated by the present invention. For example, FIGS. 1b-1d illustrate a plurality of lamps 58 on housing 50.

Lamp electric power source 64 provides an appropriate voltage received from socket 62 to lamp 58. Lamp electric power source 64 includes a transformer and other related electric components, as in a conventional ballast unit for a fluorescent lamp. Lamp electric power source 64 is fixedly supported by members formed on an inner wall of housing 50.

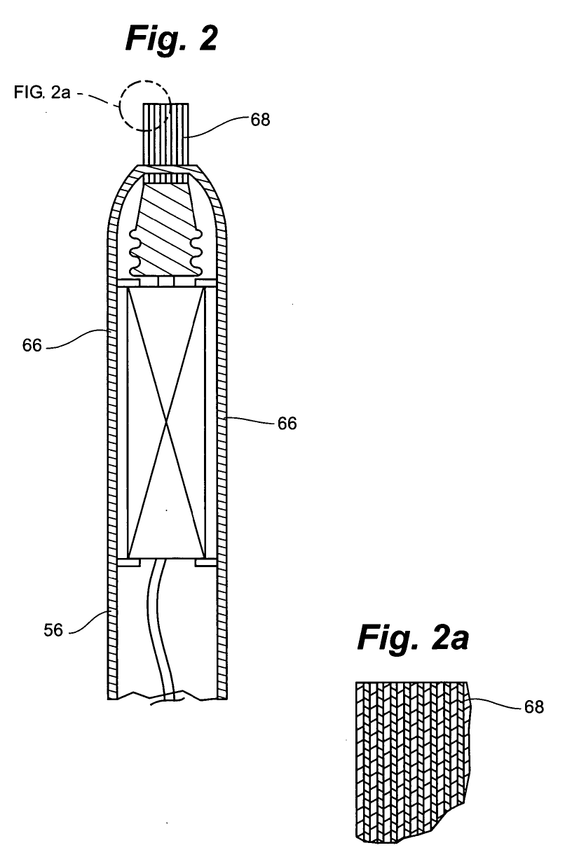

Anion generator 56 is centrally mounted on upper part 52 of housing 50. Anion generator 56 includes a metal fiber (MF3) brush 68 for generating anions without generating ozone. Brush 68 has numerous tiny projections, and each of the projections has a sharp tip. Preferably, the metal fibers may be made of a conductive alloy, and more preferably the alloy is made up of eight different metals. Most preferably, the alloy comprises platinum, stainless steel, copper, silver, zinc, nickel, manganese and tungsten. The fibers may be bound together by rope-twisting them with a conductive resin and/or a semi conductive resin. The anion generator is further illustrated in FIG. 2 which has a partial close-up view of metal fiber brush 68. An non-conductive, perforated cap 70 may be provided to fit over brush 68 and anion generator 56 to protect brush 68 and prevent accidental contact by a user. The cap 70 may be, for example, of plastic and provided with holes, slots or other perforations to permit the anions to be discharged.

As previously discussed, conventional anion generators using electronic guns generate ozone, which is undesirable, in addition to anions. In the lighting apparatus of the present invention, however, anion generator 56 comprises the metal fiber brush 68 generating anions without generating ozone. Thus, since the lighting apparatus of the present invention does not generate the air pollutant ozone and decomposes or dissolves organic substances in the air, it provides users with more purified air.

Referring to FIG. 3, the lighting apparatus of the present invention comprises a circuit 100 for receiving AC electric power, rectifying the AC electric power into DC electric power, applying the DC electric power to a ballast, and performing amplification of voltage and supply of constant voltage, thereby turning on the lamps.

Further, the circuit includes an interference protection unit 110 and an ion generation voltage induction unit 120 which provides high voltage to the metal fiber brush 68. The interference protection unit 110 receives AC electric power, and prevents the ballast and the anion generator 56 from interfering with each other. The ion generation voltage induction unit 120 converts the AC electric power applied to the protection unit 110 into DC electric power of −5 kV, and generates pulses with a frequency multiplied by an integer ratio by a diode-capacitor multiplier. The metal fiber brush 68 receives current from the induction unit 120.

As is apparent in from the above description, the lighting apparatus of the present invention purifies the air without a UV lamp. Further, the lighting apparatus does not generate significant amounts of ozone because it uses the metal fiber brush. Further, the lighting apparatus prevents anions generated from the anion generator from being moved by electromagnetic interference.

Preferably, a titanium dioxide coating layer serving as a photocatalyst is applied to the exterior of lamp 58. The titanium dioxide is activated by light rays generated from the lamp 58, so that it decomposes or dissolves organic or inorganic substances. That is, when the substances approach to or come in contact with the photocatalyst activated by the light rays, they are subjected to oxidation and/or reduction reactions, so that they are decomposed or dissolved by a photocatalyst effect. The use of the titanium dioxide coating provides disinfection and deodorization of the air in addition to the generation of desirable anions in the same compact, convenient lighting device.

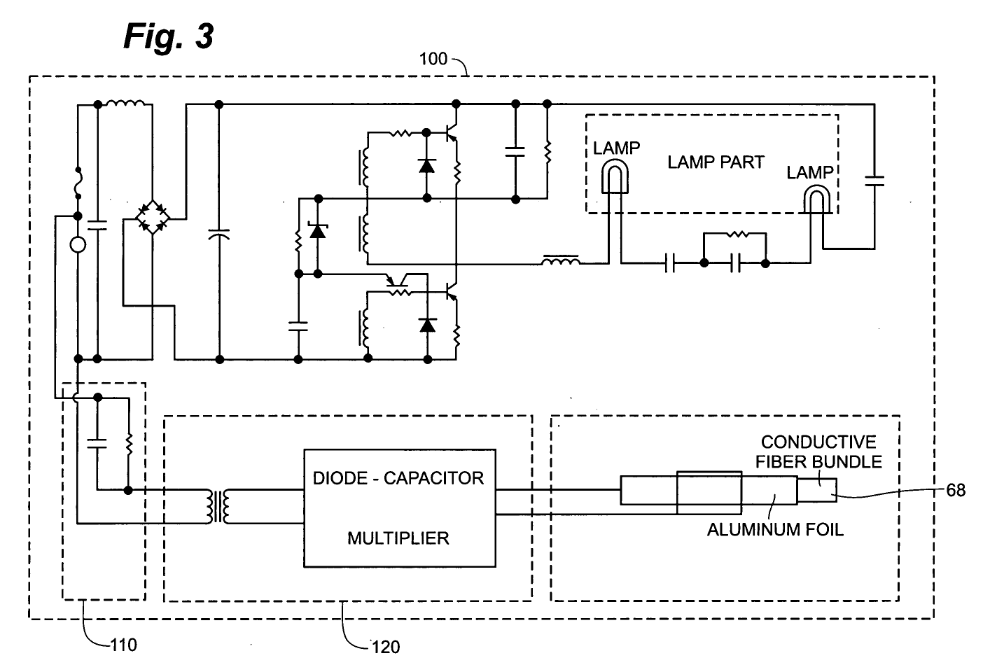

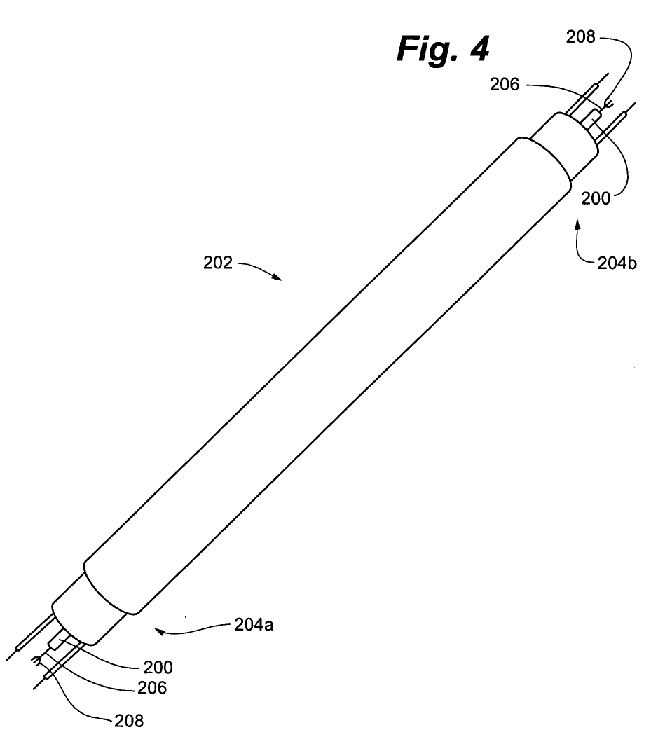

In another embodiment of invention as illustrated in FIG. 4, an ionizer apparatus 200 is incorporated into a light bulb tube 202, such as a fluorescent light tube. Fluorescent tubes are available in a variety of lengths and have different diameters which are generally referred in the industry by the “T” size (“T” standing for its “tubular” shape), e.g. T4, T5 and T8 sized bulbs. The standard industry tube is the T12 tube, the “12” standing for its diameter of 1½ (Measured in eighths of inches). The T8 tube has a diameter of 1 inch, while the T5 tube ahs a diameter of ⅝ inch. The ionizer apparatus 200 may be incorporated at one or both ends 204a, 204b of light bulb tube 202. Ionizer apparatus 200 comprises a negative ion generator 206 having a metal fiber brush 208 for generating anions without generating significant amounts of ozone. Light bulb tube 202 may also be coated with titanium dioxide to provide disinfection and deodorization of the air when the coating is activated by the light from light bulb tube 202.

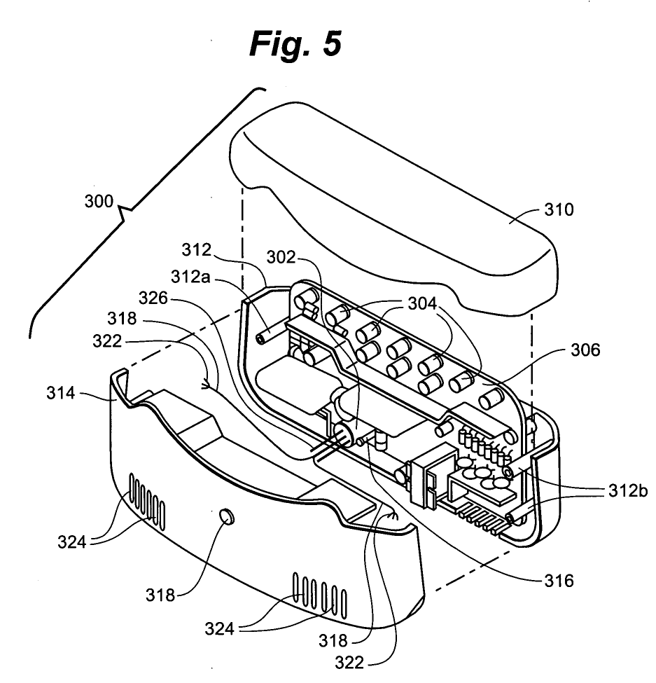

In another embodiment, an ionizer apparatus 302 is incorporated into a night light 300 as illustrated in FIG. 5. Night light 300 is provided with at least one light emitting diode (“LED”) 304. FIG. 5. illustrates a plurality of LEDs 304 mounted on a printed circuit board 306. Printed circuit board 306 is positioned in a night light body 308.

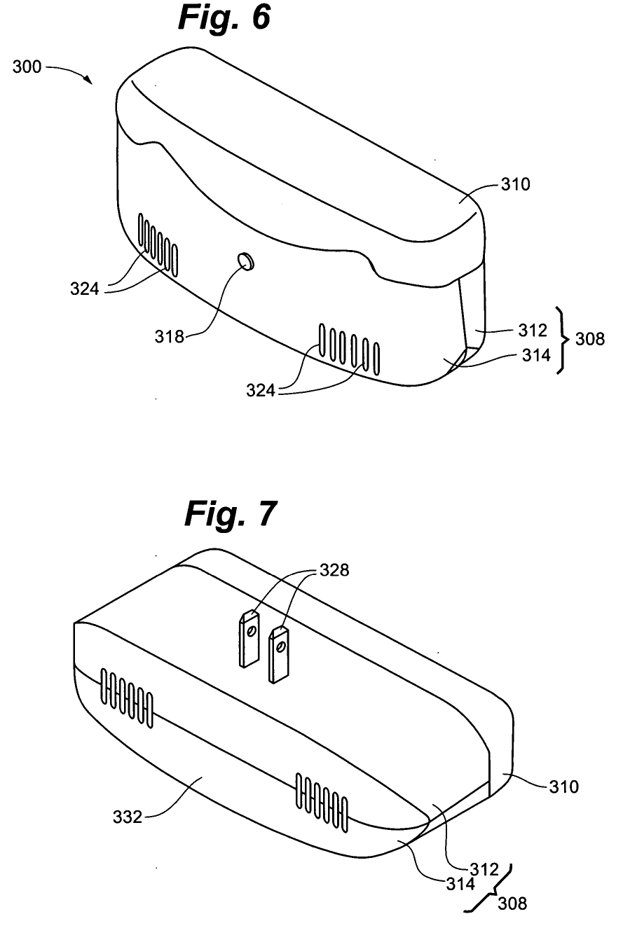

Referring to FIGS. 5 and 6, night light 300 includes a night light body 308 provided with a plurality of LEDs 304 and light transmission cover 310 coupled to night light body 308 to diffuse light radiated from the LEDs 304 and to transmit the diffused light to the outside of the night light. The night light body 308 is the main part of the night light 300 that includes a variety of electric circuits required for the control of the night light, and is formed of a pair of pieces that is easily attached to and detached from each other in view of the assembly and maintenance thereof.

Referring to FIG. 5, first and second body pieces 312 and 314, respectively, of night light body 308 can be attached to and detached from each other using bosses 312a and 312b formed in the first and second body pieces 312, 314, respectively, and screws (not shown). In night light body 308, printed circuit board 306 on which various electric circuits and a microprocessor or appropriate logic circuitry are mounted is placed inside the first body piece 312 in the longitudinal direction of the night light, and terminals 329 (see FIG. 7) are projected to the outside of first body piece 312 for plugging into a wall socket so as to supply power to the printed circuit board 306.

A sensor 316 protrudes from the center of the printed circuit board 306 to be inserted into sensor cover 318 of the second body piece 314. Sensor 316 detects the brightness of the surroundings around the night light body 308 and transmits a detection signal to printed circuit board 306. In this embodiment, sensor 316 is an illumination sensor, but the invention is not limited in the type of sensor incorporated in the device. For example, the sensor could be a motion sensor.

Sensor cover 318 is mounted on the center of the second body piece 314 to receive sensor 316. Sensor cover 318 is made of a transparent material to allow sensor 316 to correctly detect the brightness of the surroundings around night light 300. For example, when sensor 316 detects the brightness of the surroundings and transmits a detection signal to printed circuit board 306, the microprocessor or logic circuitry on printed circuit board 306 determines whether to turn on any one of the LEDs 30, to turn on two or more of them, or to select and operate some of them. As a result, sensor 316 functions as a switch for turning on and of the individual or combined operations of the LEDs 304. Accordingly, the night light of the present invention is more convenient than a conventional night light that the user must manually turn on and off. Furthermore, even though the user mistakenly plugs the night light into an outlet in the daytime, LEDs 304 are not turned on in response to the signal of sensor 316, so that excessive energy consumption can be reduce.

The LEDs 304 may be monochromatic or polychromatic LEDs as occasion demands, and the number of the LEDs may be varied and/or appropriately adjusted. As long as all of the LEDs do not break down, there are no cases where the night light does not operate.

In general, LEDs 304 are operated according to initial settings. When one, two or all LEDs 304 are operated and light is generated, the light is transmitted to the outside of the night light through the light transmission cover 310, thus implementing illumination. Accordingly, the night light of the present invention can be used as a bedtime lamp or guide lamp. While the LEDs are operated and are radiating light, a relatively high voltage is applied to a negative electrode 326 of ionizer 302, which is supplied with power from printed circuit board 306, so that anions are generated.

Ionizer apparatus 302 is positioned within first body piece 312 of night light body 308. Ionizer apparatus 302 comprises one, two or a plurality of negative ions generators 312 extending from an ionizer power supply (not shown). Each negative ion generator has a metal fiber brush 322 which is positioned adjacent to air slots 324 on first body piece 312.

While the anions are discharged and moved, air flowing into the night light body 308 through air slots 324 reacts with the generated anions and is converted into anions (i.e., the number of anions is increased). During the repeated performance of operation, numerous anions are generated in the air surrounding the positive electrode 328 of ionizer 302. The generated anions are discharged through anion discharge holes 330 on the lower surface 332 of night light body 308, so that the night light functions to discharge anions as well as to perform illumination.

In accordance with the night light of the present invention, power consumption can be reduced, the life span of component lamps can be lengthened and the costs of maintenance can be reduced by replacing an existing incandescent bulb used as light sources with the LEDs. If such a night light can be used as a bedtime lamp, the brightness of the surroundings around the night light can be automatically detected and the LEDs are operated based on the detection of the brightness, so that the convenience of the night light is improved.

Furthermore, by controlling the kinds and number of colors of the LEDs 20 to be mounted on the printed circuit board and the amount of voltage applied to the LEDs, the atmosphere of a room caused by the light of the night light can be improved.

The nightlight is not limited to the shapes illustrated in the drawings, and may be also manufactured in a variety of other shapes and sizes without detracting from the places of installation and use of the nightlight.

It is also contemplated that an ionizer may be incorporated into a wide variety of appliances and common household items, so that an ionizer may be conveniently located and may draw power from the same source as the appliance, whether that appliance is plugged into an AC outlet or is battery powered.

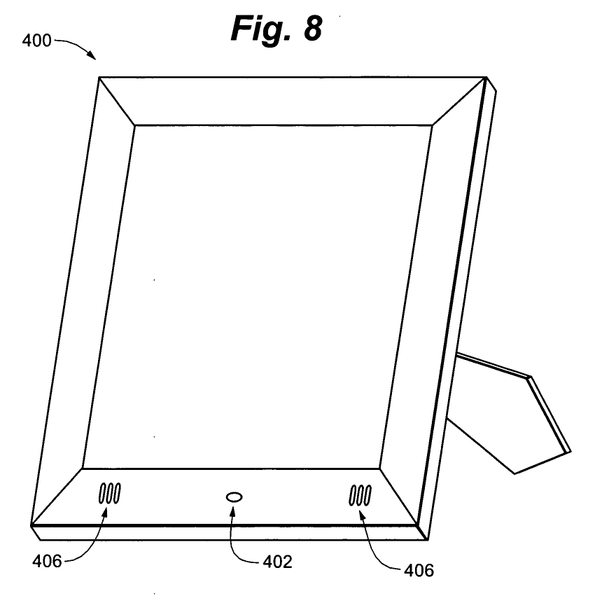

For example, an ion generator may be incorporated inside a photograph frame 400 as illustrated in FIG. 8 Frame 400 may be any conventional frame being of a shape and size to accommodate photographs therein for display on a wall or surface such as desk or table. Frame 400 may also be provided with at least one LED on a printed circuit board (not shown) positioned on the interior of frame 400. Frame 400 may also include a light sensor 402 which will cause the LEDs to illuminate the frame when the room is darkened. Negative ions generated by the ionizer are emitted from frame 400 via air slots 406. The metal brush of the ion generator is positioned inside the frame 400 adjacent to air slots 406.

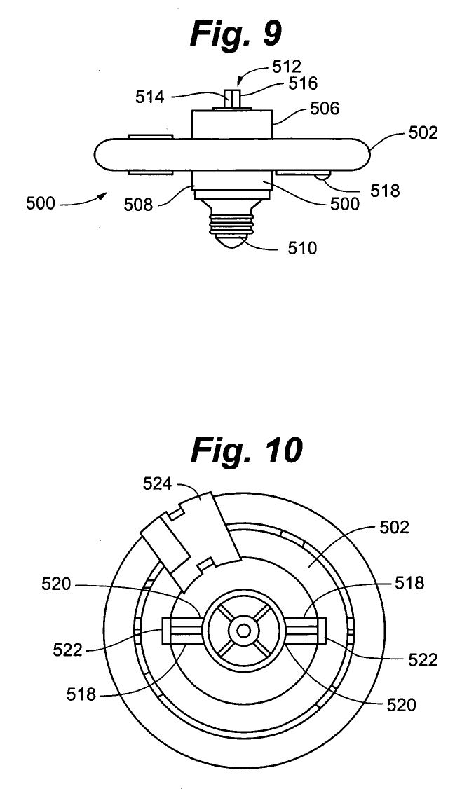

In another embodiment, a ballast 500 for retaining an operating a circular light tube 502 is provided as illustrated in FIGS. 9 and 10. Ballast 500 is comprised of a housing 504 having an upper end 506 and a lower end 508. A negative ion generator 512 is positioned inside housing 504, and a metal fiber brush 514 of negative ion generator 512 extends out of a protruded portion 516 on top end 506 of housing 504.

Ballast 500 is provided with at least one, and usually two or more, arms 518 which extend outward from housing 504. Arms 518 have a proximate end 520 and a distal end 522. Distal ends 522 of arms 518 are configured to receive and removably retain a circular bulb 502 in a semi-circular retainer element. The retainer elements generally hold circular bulb 502 by “gripping” a portion of circular light tube 502. The circular tube 502 is easily removable and replaceable by pulling so that circular light tube 502 is easily released form the retainer elements.

Ballast 500 also includes a plug 524 that is connected at one end to a power supply (not shown) positioned within the ballast 500. Plug 524 at its other end is connected to circular bulb 502 to provide power for illumination. The invention is not, however limited to a ballast for holding a circular light tube, and other tube shapes are contemplated. Bulb 502 may be provided with a photocatalyst coating such as titanium dioxide.

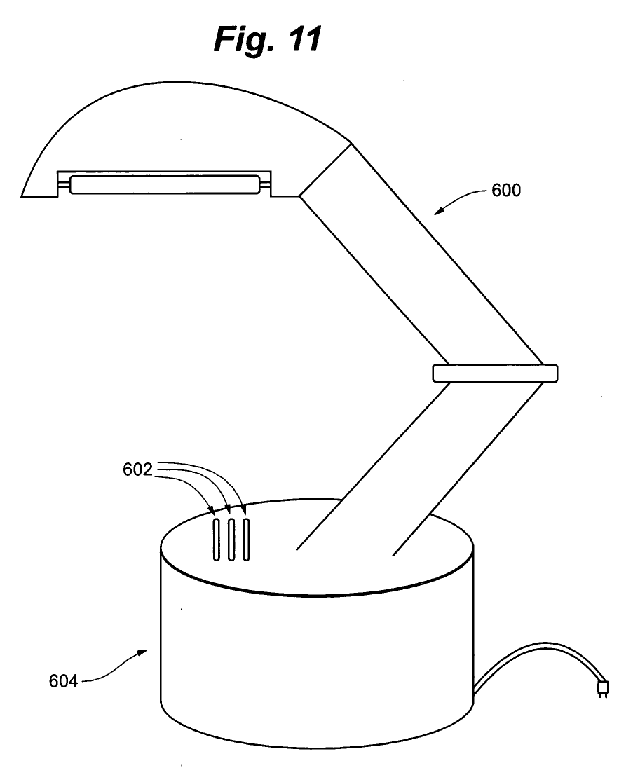

FIG. 11 illustrates a desk lamp 600 within which an ionizer may be positioned for emitting anions into a room or space via air slots 602. In FIG. 11, the ionizer is positioned inside the base portion 694 of lamp 600. The ionizer position is not limited in this respect and may be positioned in other parts of the lamp.

With regard to the light bulbs, tubes and lamps, as described herein, the present invention is not limited to the type of light emitted therefrom, and encompasses full spectrum light, compact fluorescent light, incandescent light and light of other sources and wavelengths.

Because of the compact size and relatively low power requirements of the ionizer of the invention, the anion generator may be incorporated into a wide variety of appliances and other household items. This allows the ionizer to be conveniently and aesthetically placed in a room, and the ionizer may share a power source with the appliance.

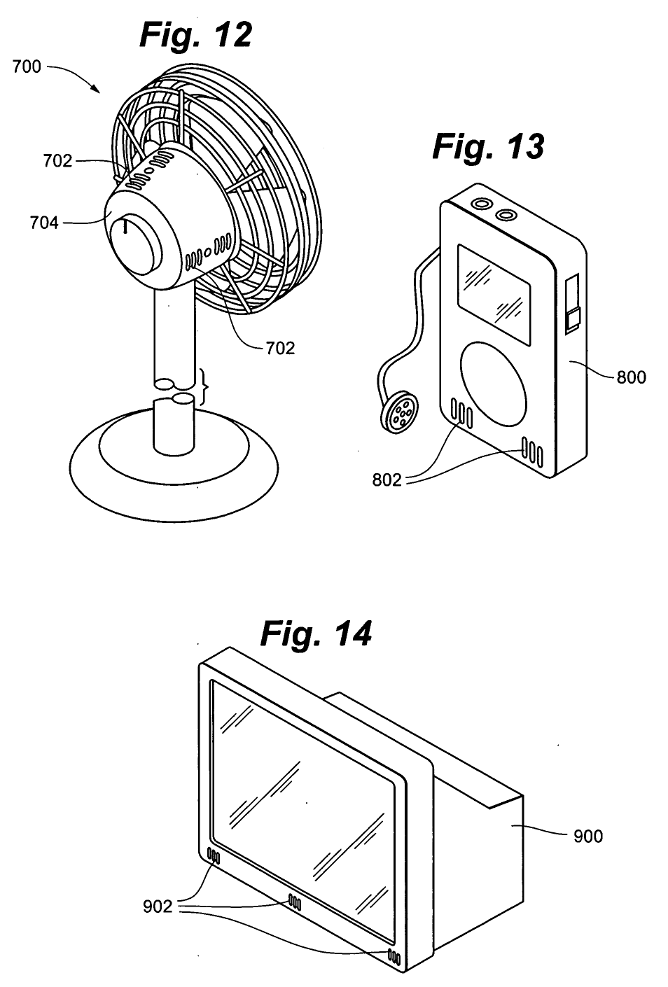

By way of illustration, as shown in FIG. 12, an ionizer may be incorporated into a fan 700. Slots 702 in housing 704 permit anions to be emitted. This embodiment has the additional advantage that the emitted anions are dispersed into the ambient air by the fan.

As shown in FIG. 13, the ionizer may be incorporated into a portable entertainment device such as a portable audio player 800 like an MP3 player, CD player, tape player or radio. Slots 802 allow the ions to be emitted. Since a device of this type is often worn on a person's belt or on an armband, the ions are released into the individual's personal space. Player 800 and the ion generator therein are powered by batteries, either disposable or rechargeable.

FIG. 14 illustrates a television set 900 including slots 902 to permit emission of ions from an ion generator located inside the set.

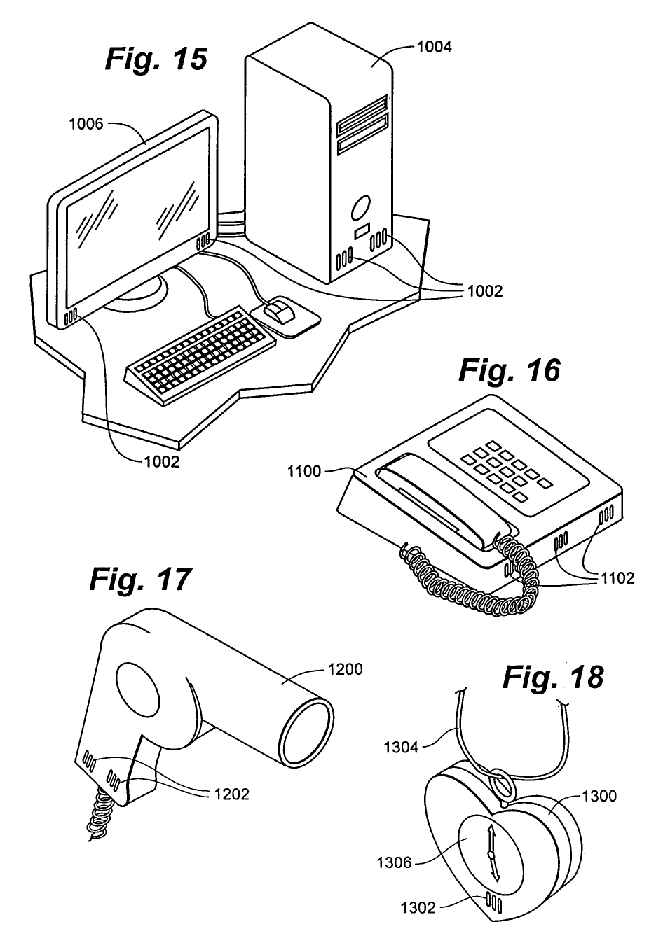

FIG. 15 illustrates a personal computer 1000 having an ion generator located in the CPU case 1004 and/or the monitor 1006. Slots 1002 permit the discharge of ions into the air around computer 1000. Although FIG. 15 shows a desktop computer, the ion generator of the invention may also be incorporated into a portable (laptop) computer or personal digital assistant (PDA) device.

FIG. 16 depicts a telephone 1100 with slots 1102 to permit emission of anions from an ion generator inside the telephone. Although FIG. 16 shows a desktop telephone, the ion generator of the invention may also be incorporated into a cordless, wireless or cellular telephone.

FIG. 17 shows a hair dryer 1200 with slots 1202 to permit emission of ions from an anion generator within the hair dryer.

FIG. 18 shows a pendant 1300 having an ion generator therein. Slots 1302 allow the release of anions into the user's personal space. Pendant 1300 may be worn as a necklace on chain 1304, and may include a small clock 1306. An ion generator may also be built into a wristwatch.

The anion generator of the invention may also be advantageously installed in the passenger compartment of an automobile, for example, in the dashboard, and draw power from the vehicle's electrical system. Auto interiors often suffer from poor air quality due to vehicle exhaust, cigarette smoke, VOCs from upholstery and the like, and the anion generator of the invention can greatly enhance air quality in this enclosed environment.

Additionally, each of the apparatuses described herein may further include a scenting device incorporated within for emitting pleasant scents or fragrances from the apparatus.

Although the preferred embodiments of the present invention have been disclosed for illustrative purposes, those skilled in the art will appreciate that various modifications, additions and substitutions are possible without departing from the scope and spirit of the invention as disclosed.

We claim:

1. A lighting apparatus comprising:
an anion generator comprising a metal fiber brush;
an illumination unit; and
a power source for supplying electric power to the ion generator and the illumination unit.

2. The lighting apparatus of claim 1, wherein the metal fiber brush is made of a metal alloy comprising platinum, stainless steel, copper, silver, zinc, nickel, manganese and tungsten.

3. The lighting apparatus of claim 1, wherein the metal fiber brush comprises a plurality of sharp protrusions.

4. The lighting apparatus of claim 1 wherein the illumination unit comprises a fluorescent tube.

5. The lighting apparatus of claim 4, wherein the illumination unit is coated with a photocatalyst.

6. The lighting apparatus of claim 5, wherein the photocatalyst is titanium dioxide.

7. The lighting apparatus of claim 1 wherein the illumination unit comprises at least one light emitting diode.

8. An air purifying apparatus comprising:
a housing;
an ion generator for purifying air mounted within said housing, said ion generator comprising a metal fiber brush for generating anions; and
at least one aperture in said housing for emitting anions therethrough.

9. The apparatus of claim 8 wherein the housing comprises a picture frame.

10. The apparatus of claim 8 further comprising a fan.

11. The apparatus of claim 8 further comprising a portable audio player.

12. The apparatus of claim 8 further comprising a television set.

13. The apparatus of claim, 8 further comprising a computer.

14. The apparatus of claim 8 further comprising a telephone.

15. The apparatus of claim 8 further comprising a hair dryer.

16. The apparatus of claim 8 further comprising a clock.

17. The apparatus of claim 8 wherein the housing is a pendant adapted to be about a user's neck.

18. The apparatus of claim 8 wherein the housing is adapted to be mounted within a passenger compartment of an automobile.

19. A method of providing air purification using a lighting apparatus having at least one anion generator comprising a metal fiber brush and at least one illumination unit, comprising the steps of:
supplying electric power to the apparatus;
amplifying the electric power and applying the amplified power to the anion generator;
generating anions by the metal fiber brush of the ion generator; and
discharging the generated anions from the apparatus to purify the surrounding air.

20. The method of claim 19 wherein the illumination unit is a fluorescent tube and comprising the further steps of;
providing a photocatalyst coating on the fluorescent tube; and
activating the photocatalyst coating by illuminating the tube.
